(19) United States
(12) Patent Application Publication
(10) Pub. No.: US 2011/0153008 A1
(43) Pub. Date: Jun. 23, 2011

(54) ARTIFICIAL VALVE

(76) Inventors: Coralie Marchand, Villeurbanne (FR); Frederic Heim, Hattstatt (FR); Bernard Durand, Pfastatt (FR); Nabil Chakfe, Eschau (FR); Jean-georges Kretz, Strasbourg (FR)

(21) Appl. No.: 12/733,155

(22) PCT Filed: Aug. 4, 2008
(86) PCT No.: PCT/FR2008/051456

§ 371 Date: Mar. 22, 2010

(30) Foreign Application Priority Data
Aug. 9, 2007 (FR) 0757012

(51) Int. Cl. A61F 2/24

(52) U.S. Cl. 623/2.19

(57) ABSTRACT

The invention relates to a valve endoprosthesis, characterised in that it substantially comprises an extensible stent or frame (1) made of several parts, i.e. an upper cylinder (11), a lower bearing portion (21) having the shape of a truncated cone and a maximum diameter higher than that of the aortic ring and decreasing down to the diameter of the extensible stent or frame (1) in the direction of the proximal end, and arches (31), the upper cylinder (11) being connected to the lower bearing portion (21) via mounts (41) and by a valve (2) connected to said stent (1) by stitches, staples or clips. The invention can particularly be used in the field of medicine, in particular in plastic surgery, and particularly in cardiac surgery, in particular for cardiac prostheses.

The present invention concerns the field of medicine, in particular plastic surgery, and especially heart surgery, more specifically cardiac prostheses, and its object is an artificial valve.

The human heart functions like a pulsed-flow pump whose main function is to create blood circulation in the veins and arteries in order to supply oxygen and various nutrients to the organs of the body that need them. To ensure a continuous blood flow, it is essential that there is no blood reflux, that is, that the blood does not flow backwards during the non-pumping or relaxation phases of the heart muscle. For this purpose, the heart is equipped with heart valves that act as check valves. These valves can become defective with time, however, and may require replacement by prostheses, which at present still do not completely recreate the very complex physiology of the individual.

The implantation of artificial aortic valves is generally necessary to make up for deficiencies due to the degeneration of the aortic valve, more specifically for reasons of calcification of the valve tissue due to abnormal impregnation of the tissues by calcium salts as a result of degeneration of the collagen fibers that constitute that tissue. The result is rigidity of the valve tissue and a loss of flexibility during the movement of the cusps caused by the action of the pumping heart. These cusps forming the valve are in effect subjected to a continuous movement in the opening and closing of the aorta at a rate corresponding to the heart rate, so that any rigidity of the tissue comprising them irremediably causes accelerated wear by fatigue.

This wear of the heart valve has two types of consequences: symphysis of the valve, creating aortic narrowing and thus an obstacle to left ventricular ejection, and destruction of the valve, creating diastolic reflux into the left ventricle. These two effects lead to heart failure.

The patient thus experiences stenosis, that is, partial blockage or narrowing of the aortic channel due to the fact that the complete opening of the valve is prevented. This type of pathology develops mainly in older subjects.

In addition, in an affected subject, particularly one with exaggerated fatty deposits, a particular physiological reaction consists of the formation of blood clots, or thrombosis, which is the result of the deposition of fibrin and platelets in the pathological areas. Such clots impede the movement of the valve leaflets, which can no longer completely close the valve, leading to aortic regurgitation or valve leaks. Other types of degeneration may also occur, such as tissue ruptures, which may be another source of valve dysfunction.

To prevent these problems, artificial replacement valves have been developed beginning in the mid-twentieth century, in particular with the appearance of mechanical valves. During the period from roughly 1960 to 1970, biological valves were developed. Thus there are a number of satisfactory artificial replacement valves currently available, even though the optimum artificial valve, that is, one that can satisfy all physiological requirements, does not yet exist.

To this effect, the first methods used to make mechanical valves involved the formation of a ball of biocompatible material housed in a cage simultaneously forming a leak-tight seat on the side toward the heart muscle, or the formation of one or two disks joined to a frame. These valves were implanted at the outlet of the heart muscle, in the end of the artery containing blood reflux sinuses during the closing of the valve.

These known mechanical valves have an excellent functional reliability and a long lifespan on the order of 25 to 30 years, but they cause turbulence that may be responsible for producing a phenomenon which creates a risk of thrombosis such that patients who have had them implanted must take an anticoagulant for life. This obstacle is also responsible for a pressure gradient, which requires additional effort from the heart muscle.

To avoid these disadvantages, the use of biological valves has been proposed, that is, artificial valves made from organic tissues, either human (allografts) or animal (xenografts). These biological valves, which are a common solution for the replacement of defective natural valves, recreate human physiology by allowing a central flow and are generally very well tolerated and offer the patient a good quality of life while also eliminating the need for anticoagulants.

This last point is especially important in the case of patients for whom taking anticoagulants is not recommended, that is, older patients or pregnant women. Moreover, these artificial valves are especially suitable because they offer no resistance to central flow and have a better resistance to thrombosis than the mechanical valves.

Because they are made of organic tissues, however, they are subject to aging and natural degeneration and their lifespan is limited to about 10 to 12 years, so that a new operation is necessary in 74% of cases.

Artificial valves made of synthetic material have also been proposed, in particular those made of polyurethane or molded silicone. Nevertheless, these valves have fatigue resistance problems, with a risk of rupture in the areas of flexion.

Finally, EP-A-1 499 266 discloses a method for making an artificial aortic or mitral valve that consists essentially of shaping an artificial valve of a textile material. Such an artificial valve makes it possible to avoid taking anticoagulants (the geometry reproduces that of the natural valve) while avoiding the degeneration characteristic of biological tissue. It is completely biocompatible and has an excellent resistance to aging.

In recent conferences, specialists set a short-term objective of generalized aortic valve replacement by the percutaneous route. To date, such a procedure is still at an experimental stage, with only some hundred implantations having been performed worldwide with no significant inroads having been made.

The appeal of the new procedure is that it is a noninvasive surgery avoiding a serious operation for the patient, that is, it avoids the opening up of the chest and stopping the heart as is the case with a traditional heart valve implantation. Because of the lengthening life expectancy in the population, aortic valve replacement will involve an increasing number of older and therefore at-risk people. In addition, the cost associated with a valve replacement is significant because of the infrastructure associated with the operation itself and because of the rehabilitation of the patient that is necessary.

A first biological valve implantation by the percutaneous route was thus carried out in 2002 and has since been followed by about a hundred others. In these cases, the biological valve was associated with a traditional cylindrical arterial stent or extensible frame. In the rest of the specification, we will use only the term stent for the sake of simplicity.

The results obtained with these new artificial valves can be considered satisfactory, since the patients in question had pathologies that would not have tolerated another type of intervention.

In certain cases, however, the implantation led to migration of the stent because its anchoring in the aortic root was not satisfactory. Moreover, problems of poor positioning, infection, mitral and coronary disturbances, and valve leaks were also found. To overcome these disadvantages, a certain number of valved stents have been developed and proposed. Artificial valves are currently essentially tubular and reproduce the geometry of arterial stents and can be classified into three categories of devices: short-tube, medium-tube, and long-tube.

The short-tube devices have essentially two types of implementation, that is, positioning with a significant radial stress and by means of hooks, or positioning by inflation and polymer injection.

In the first case, the positioning technique is completely mastered, but this positioning is uncertain at the height of the aortic ring and the radial stress is damaging to the tissues. In addition, an angular positioning cannot be made, the seal is dependent on the radial stress, and there is a high risk of migration.

In the case of positioning by inflation, the geometry of the aortic root is perfectly matched, so that the seal is ensured. Moreover, this procedure is not damaging for the valve or for the tissues. Nevertheless, positioning also remains uncertain at the height of the aortic ring, angular positioning is not ensured either, and partial blockage of the sinuses occurs.

The medium-tube devices also feature two types of implementation—one involves positioning with a significant radial stress with a long stent and hooks, and the other involves positioning by pinching the natural valve.

In the first case, the positioning technique is completely mastered and positioning is performed by the length of the stent. Nevertheless, the radial stress is damaging to the tissues, the seal is dependent on the radial stress, and there is a risk of migration. In addition, angular positioning is not possible.

In the positioning solution involving pinching the natural valve, positioning is done by the stent length and in an angularly defined way, avoiding the risk of migration. But the radial and pinching stress is damaging to the tissues, and the seal is entirely dependent on this stress. In addition, blood flow as well as the mitral valve are negatively affected.

The long-tube devices are generally positioned with a significant radial stress and making use of the stent length and moreover have the advantage of avoiding the risk of migration as well as achieving good positioning due to the stent length. Some of these devices also allow for angular positioning in the sinuses. In these devices, however, there is still the disadvantage of a radial stress damaging to the tissues and possibly having a negative impact on the seal. In fact, the use of hooks is traumatic for biological tissues, which, because of significant radial stress, can sustain damage that compromises the good functioning of the artificial valve over time. Moreover, in some of these devices there is a negative effect on blood flow and the mitral valve, while for others the positioning is too high in the aortic root.

Another device has also been developed and is positioned by the stent length and with an obstacle in the sinuses. This device allows for good positioning because of the stent length and avoids the risk of migration thanks to the obstacle in the sinuses, while guaranteeing angular positioning in the sinuses and the reshaping of these latter. In addition, this device does not negatively affect the mitral valve.

The radial stress necessary for the seal of the device is damaging to the tissues, however. Finally, the coupling between the upper and lower parts of the stent requires a certain sinus height, so that adjusting to variable patient morphology is not possible.

Moreover, from WO-A-2005/046528 we know about an artificial valve whose lower part is flared. This geometry is characterized by a progressive increase in the diameter of the lower part of the body of the device toward its bottom end. Thus the support of the lower, flared part in the natural channel is achieved by means of a line of contact in the aortic sinuses. The sinuses are made of elastic and very deformable tissues, and the support of the lower, flared part of the stent upon these is limited to one line of contact, which itself causes significant stresses. The sinuses are consequently highly deformed locally, which increases the risk of a possible disturbance in blood flow. This method of anchoring does not make it possible to keep the aortic environment intact.

In addition, the lower, flared part whose contact with the wall of the aortic sinuses is reduced to a discontinuous line of contact does not make it possible to ensure the seal of the device because it does not adapt to imperfections in the aortic ring.

Finally, EP-A-1 690 515 describes a device equipped with arches extending outward relative to its diameter, against the walls of the aortic sinuses, thus ensuring the positioning of the artificial valve. These arches should ensure contact with the wall of the sinuses, in order to ensure the anchoring of the artificial valve and leave blood flow undisturbed. Since, however, the aortic sinuses are subject to changes in size over the course of the cardiac cycle, the artificial valve must therefore and in any case adjust to these changes in size in order to ensure contact of the arches on the sinus wall. Such flexibility in adjusting to the morphological variations in the aortic sinuses is not specified in this document, however.

Neither of these two documents presents an artificial valve device with a distal or proximal means of anchoring.

The goal of the present invention is to eliminate the disadvantages of the new artificial valves described above by proposing an artificial valve allowing on the one hand for percutaneous implantation, and on the other hand making it possible to avoid the problems of deterioration of the prosthetic material and human tissues as well as to ensure the seal of the device, while ensuring at the same time that it is also maintained in position at the implantation site and that the implanted artificial valve functions properly.

For this purpose, the artificial valve is characterized by the fact that it essentially consists of: a stent or extensible frame consisting of several parts, that is, an upper cylinder, a lower support part in the shape of a truncated cone whose maximum diameter is greater than that of the aortic ring and decreases to the diameter of the stent or extensible frame in the direction of the proximal end, and arches, whereby the upper cylinder is connected to the lower support part by means of struts, and a valve connected to the stent by sutures, staples, or clips.

The invention will be better understood thanks to the description below, which concerns a preferred embodiment, given as a non-limiting example and explained with reference to the attached schematic drawings, in which:

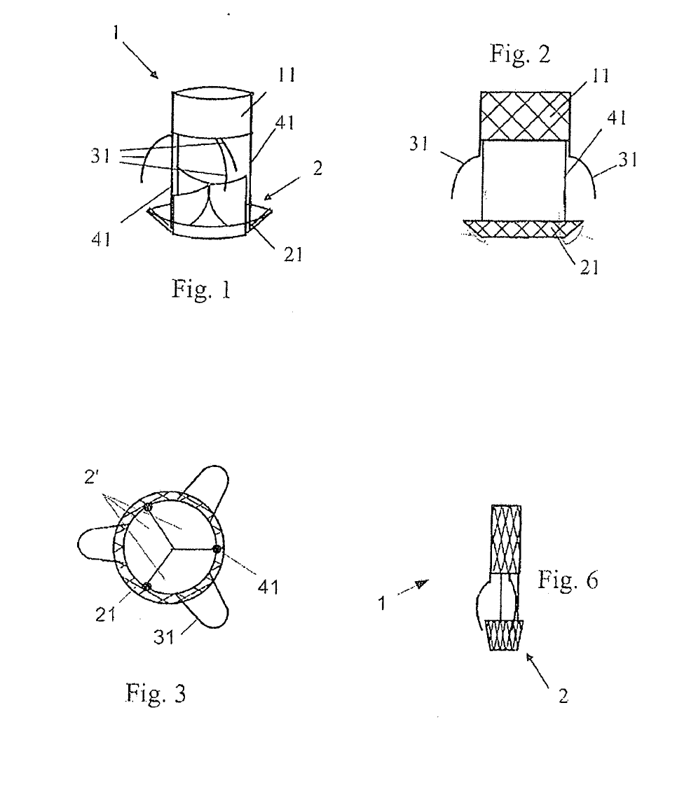

FIG. 1 is a perspective view of the artificial valve according to the invention;

FIG. 2 is a side elevation of the stent or extensible frame of the artificial valve according to FIG. 1, without the textile valve;

FIG. 3 is a top view of the stent according to FIG. 1;

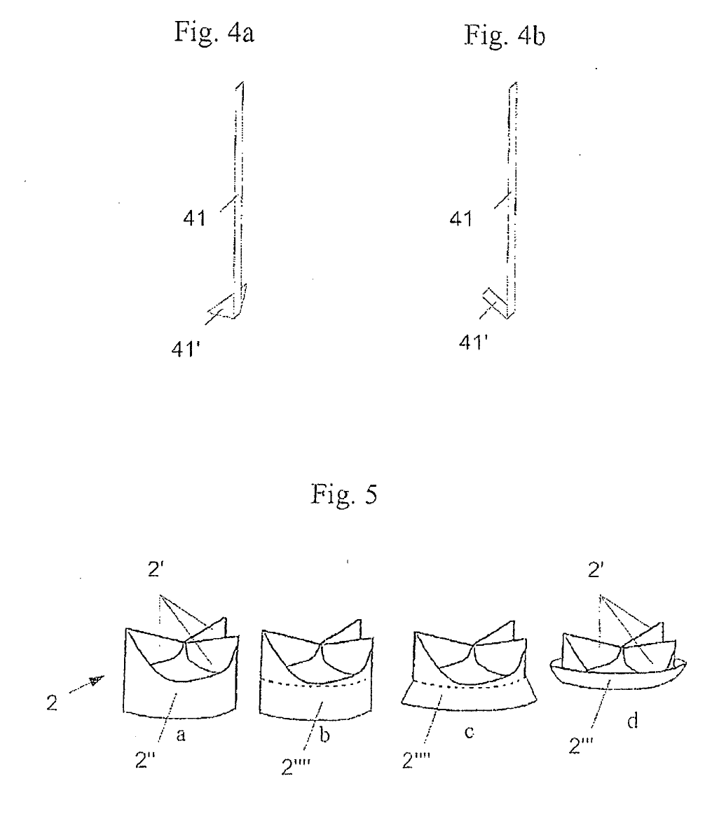

FIGS. 4a and 4b are partial perspective views showing the struts connecting the upper part of the stent to its lower, conical part;

FIGS. 5a to 5d show successive configurations of the textile valve for the purpose of mounting it on the lower, conical part of the stent; and

FIG. 6 shows the stent in compressed position before it is put in place.

FIG. 1 in the attached drawings shows an artificial heart valve designed for percutaneous implantation.

According to the invention, this artificial valve essentially consists of: a stent or extensible frame 1, preferably consisting of several parts, that is, an upper cylinder 11, a lower support part 21 in the shape of a truncated cone whose maximum diameter is greater than that of the aortic ring and decreases to the diameter of the stent or extensible frame 1 in the direction of the proximal end, and arches 31, whereby the upper cylinder 11 is connected to the lower support part 21 by means of struts 41, and a valve 2 connected to the stent 1 by sutures, staples, clips, or other means.

Preferably, the proximal end of the lower support part 21 forms a partially spherical or toroidal surface. Contrary to the devices known to date, the lower support part 21 thus has a progressive decrease in diameter of the lower part of the body toward its bottom end, thus offering a contact surface and not a line of contact. This contact surface rests on the aortic ring and not in the sinuses, which are not affected by contact with the lower, conical part.

According to one characteristic of the invention, the arches 31 are connected to the upper cylinder 11 and advantageously extend outward relative to the diameter of this latter. Thus the arches 31 may be flexible and may follow the deformation of the sinuses during the cardiac cycle, so as not to stiffen the sinuses as well as to minimize the stress on the tissues.

It is also possible to attach the arches 31 to another part of the stent or extensible frame 1, that is, at the top or bottom of this latter, or else to the struts 41. Of course, all derived solutions are possible, so long as the arches 31 can adjust to the sinuses, whether under static conditions in the case of a non-ideal morphology of the aortic root, or under dynamic conditions in the course of the cardiac cycle.

The arches 31 have a curved shape such that they make it possible to follow the natural shape of the sinuses, in terms of both their geometry and their support surface, which distributes the stresses and thus makes it possible not to deform the tissues locally. This configuration of arches 31 also makes it possible to avoid blocking the coronary orifices, for example by means of a refined, honeycomb-like, minimal support surface.

The artificial valve according to the invention is thus perfectly suited to implantation in natural channels with an aneurysm at the valve edges, such as an aortic root with sinuses of Valsalva, with the arches then deploying into the bulges formed by the sinuses.

The upper cylinder 11 is designed to position the artificial valve in the aorta and the sinuses in cooperation with the arches 31, while the lower support part 21 is applied against the aortic ring. As for the struts 41, they are designed to make the connection between the upper cylinder 11 equipped with the arches 31 and the lower support part 21, while ensuring a support function for the valve 2.

The positioning of the artificial valve according to the invention is ensured by the obstacles formed by the lower support part 21, whose proximal end forms a partially spherical or toroidal surface, and by the arches 31, but not by adherence as in the artificial valves proposed to date, so that the radial stress is reduced and is therefore not traumatic for the tissues. The main support is on the aortic ring and not in the sinuses.

The upper cylinder 11 and the lower support part 21 in the shape of a truncated cone are made by braiding and the arches 31 are also made by braiding and assembled by sutures with the upper cylinder 11, forming projections from this latter. Thus the braided structure of the upper cylinder 11 and the lower part 21 allows for easy elastic expansion like a grid, so that the stent 1 obtained is more flexible than if it were made of a solid, machined material. Of course, the different parts of the stent or extensible frame 1 can be more or less independent, that is, be made singly or in blocks. In addition, these different parts may also be obtained by machining, knitting, or other means.

Preferably there are three arches 31 arranged at regular intervals in the lower part of the upper cylinder 11. But it is possible to have the arrangement and number of arches of the stent such that the stent 1 is specifically tailored to the morphology of the aortic root into which the device will be implanted.

It is also possible to have the stent or extensible frame 1 be a single piece made of metal alloy, such as braided or machined Nitinol, obtained by preliminary cutting and shaping.

For this purpose, the stent 1 is made from a cylindrical or slightly conical blank into which feet are cut out at the height of the arches, which are then connected to the rest of the device only at the top. The curved geometry of the arches is then achieved by a new shaping. The struts consist of the rest of the material remaining on either side of the cutouts and are a continuous part of the upper cylinder and the conical base.

FIG. 6 in the attached drawings shows a stent 1 in its compressed position before being put in place, a position in which the arches 31 are found in a folded position very close to the cylindrical body of the rest of the stent 1. During the expansion of the stent 1 into its position shown in FIGS. 1 to 4, these arches 31 are positioned by oblique expansion in the aortic sinuses. Because of this, the sinuses, which consist of three pockets located behind the valve leaflets like three projections from the aortic tube, form receptacles receiving the arches 31. These sinuses take part in the valve closing mechanism from the standpoint of the fluid dynamics, and make it possible to ensure an axial bilateral configuration parallel to the lower support part 21, by constituting three axial support points for the arches 31.

Thanks to its design and the assembly of its different components, the stent or extensible frame 1 is therefore compressible, which constitutes a considerable advantage with respect to its capacity for percutaneous implantation. In addition, the assembly of the different components makes it possible to achieve a constant length of the stent in its deployed and compressed states. The result is that the length of the compressed stent is not increased, which facilitates its passage through the natural channels, and the positioning of the artificial valve by medical imaging at the implantation site is facilitated as well, since the final length is equivalent to the deployed length of the stent 1.

The valve 2 preferably consists of a textile membrane made of woven material, non-woven material in the form of assembled fibers, or non-woven material obtained by autofibrillation of a membrane by drawing and knitting, for which shaping is done by concentric sheathing, three-dimensional weaving, flat sheathing, cutting out by routing, and fixation and possibly thermofixation, preliminary mechanical deformation, and application of a coolant at the deformation sites, by application to a shaping part by suction effect through said shaping part and thermofixation by supplying hot gas or air drawn through the textile membrane into the shaping part or by coolant pressing the textile membrane against a shaping part. The composition and shaping of a textile valve comparable to the textile valve 2 are described in EP-A-1 499 266.

According to a variant of embodiment of the invention, the valve 2 may also consist of another flexible material, that is, biological, synthetic, or metallic.

The valve 2, shown more specifically in FIGS. 5a to 5d, advantageously conforms identically to the aortic valve and is provided with cusps 2′ on the one hand and on the other hand with a circular skirt 2″, whereby this circular skirt 2″ bears the cusps 2′ at the top and is folded like a conical dish 2′″, partially spherical or partially toroidal, along a fold line 2″″ (FIG. 5d).

According to a characteristic of the invention, the valve 2 is connected to the lower support part 21 of the stent 1 by fitting at the bottom into this lower support part 21, having its edge folded inside the edge of the lower support part 21, and being assembled together with this latter by sutures, staples, clips, or other means (FIGS. 1 and 3). It is also possible not to have a folded edge of the valve inside the edge of the lower support part of the stent 1, for example in order to limit the space occupied in the catheter and further improve the compressibility of the lower support part of the stent 1.

The struts 41 (FIGS. 1 to 4) are formed into one piece with the lower support part 21 and the valve 2, by resting on the edge folded inside the lower support part 21 by means of feet 41′ jutting out laterally and slanting upward, and by sutures, staples, clips, or other means attaching them to the assembled lower support part 21 and valve 2. These struts 41 are attached at the top to the inside of the upper cylinder 11 of the stent 1. According to a variant of embodiment, however, the struts 41 can also be attached to the outside of the upper cylinder 11. It is thus possible, with this type of attachment of the struts 41, to have a stent 1 whose compression is unimpeded and which can be compressed without increasing its length.

According to a variant of the invention, the struts 41 can also form one piece with the lower support part 21, by being made as a single unit with this latter. Moreover, the struts 41 can be flexible or rigid and made of any material, that is, metallic or synthetic.

According to another characteristic of the invention, not shown in the attached drawings, the struts 41 may have a special surface preventing the valve 2 from sliding, that is, holes, an interlacing of strands forming a ladder, texturing, or sheathing with a textile material, etc. Thus it is possible to minimize in particular the problems of friction of the valve tissues on the structure of the stent 1 and facilitate assembling the junctions of the artificial valve on the struts 41.

These struts 41 make it possible first and foremost to assemble the top and bottom of the stent 1. In addition, these struts 41 make it possible to support the junctions of the artificial valve and thus ensure its functioning by reducing the stress applied to the junctions during systole.

Thus a certain suppleness/flexibility of the struts 41 can be useful to the functioning of the artificial valve by allowing deformation by curvature, so that in the presence of an aorta larger than the aortic ring, the struts 41 will have a curvature tending to push their upper end outward relative to the lower support part 21, while in the opposite case, this curvature will have the effect of pushing the upper end of the struts 41 back inward relative to the lower support part 21.

During the implantation of the artificial valve, the deployment of the upper cylinder 11 into the aorta ensures axial guiding of the stent. The arches 31 ensure angular positioning of the artificial valve by expanding into the top of the sinuses, as well as ensuring axial positioning in the aortic root by pressing the lower support part 21 onto the aortic ring. It is thus possible to have complete control over the position of the artificial valve during its implantation, and, in particular, to position the upper cylinder 11 in the aorta, as well as to position the arches 31 in the sinuses and the lower support part 21 on the aortic ring, with a stress of radial expansion in the aorta that is relatively low and therefore not traumatic for the tissues while still sufficient to ensure the vertical attachment and vertical and angular orientation.

There are advantageously three struts 41 arranged equidistantly around the periphery of the lower support part 21. It is also possible, however, according to a variant of embodiment of the invention not shown in the attached drawings, to equip the stent 1 with six struts 41, whereby three of these struts ensure that the upper cylinder 11 and the lower support part 21 are equidistant, and the other three ensure the attachment of the valve 2 to the lower support part 21. In such an embodiment, the struts ensuring the attachment of the valve 2 extend inside the upper cylinder 11, possibly without guiding contact with the inside wall of this latter, while the three struts ensuring the attachment of the valve 2 can be flexible or rigid.

According to another characteristic of the invention, the struts 41 can be directly integrated by their lower end into the valve 2 during the production of this latter, in the form of a metallic wire or other means, thus creating a textile composite. Such an embodiment is especially useful for interchangeability of the valve in case of this latter's deterioration, which can be achieved without removing the upper cylinder 11 of the stent 1.

The lower support part 21 rests on the aortic ring or base of the aortic root over a broad contact surface analogous to the deformation of a flexible cone under the pressure of a ball, that is, with a linear contact that is circular or roughly circular or like the segment of a sphere. Thus contact is always ensured, regardless of uncertainties regarding the diameter of the aortic ring. This part 21 constitutes the main area of support, and keeping it in position does not require radial stress involving great strain on the tissues, as is the case with the devices known to date. In fact, the stent 1 has a lower support part 21 that ensures the proximal anchoring of the artificial valve on the aortic ring without needing to exert radial stress. The stent behaves like an obstacle in the aortic root and cannot move.

Moreover, this lower support part 21 ensures the seal by jamming the conical dish 2′″, partially spherical or partially toroidal, of the valve 2 between the braided lower support part 21 of the stent 1 and the aortic ring. It also exerts a radial stress on the tissues in order, for example, to maintain the conical shape, to ensure optimum opening of the valve channel, and to deal with calcifications.

This lower support part 21 may also have a certain flexibility aimed at fitting the shape of the aortic ring, for example to fit the dilation of the aortic ring during the cardiac cycle in order to minimize trauma to the tissues and ensure continuous contact with the ring.

According to another characteristic of the invention, the upper cylinder 11 and lower support part 21 as well as the arches 31 of the stent 1 are advantageously made by interlacing metallic wires. These metallic wires can be simple metallic wires or metallic wires made from a material with shape memory. Thus the entire artificial valve has great flexibility, favoring its implantation in an environment that is most often degraded, in particular calcified and irregular.

In accordance with another characteristic of the invention, the metallic wires constituting the upper cylinder 11 and the lower support part 21 as well as the arches 31 of the stent 1 can be made of the same material or different materials. The result is that precisely because the stent 1 is made of independent parts, different materials can be used for each constituent part, allowing for optimum tailoring of the mechanical properties to each function.

In the case of a textile valve that is itself made by interlacing wires, this particular design of interlacing metallic wires makes it possible to achieve a homogeneous unit with very little exposure to wear, since the wear of the textile valve 2 on the lower support part 21 is reduced thanks to the fact that there is a very low concentration of stresses compared to the use of a manufactured support with the textile valve 2 attached to it.

According to another characteristic of the invention, it is also possible to make the upper cylinder 11 of a pre-manufactured material with shape memory. Thus the cylinder 11 forming the upper part of the stent 1 can be made of a material different from that constituting the lower support part 21 and the arches 31 and can be connected to these latter as well as to the struts 41 by gluing or welding these struts 41 to its inside wall and the end of the arches 31 to its lower end, whereby the struts 41 are connected by their lower end to the lower support part 21 by sutures, gluing, or welding.

Having the stent 1 consist of several parts also allows for easier interchangeability of the lower support part 21 supporting the valve 2 if this latter should fail and thus require a new percutaneous operation, and allows as well for interchangeability of the other constituent parts of the stent, that is, the upper cylinder 11, arches 31, and struts 41.

Thanks to the invention, it is possible to make an artificial heart valve in which the stresses on the tissues of the aortic root are minimized, since the seal is ensured by obstruction, that is, by the combination of the stent 1 and the valve 2 sandwiched in the lower support part 21 between this latter and the aortic ring, and not by using significant radial stress, thanks to the geometry of support on the aortic root and in combination with the arches, which ensure that the support is bilateral since they are positioned in the sinuses. Moreover, because the stent 1 consists of several parts, it can adapt to different aortic root morphologies, that is, different heights, with less stress on the tissues.

In addition, the flexible structure of the lower support part 21, obtained by braiding, allows for better adaptation to an aortic root that may have irregularities.

Finally, the artificial valve according to the invention can be positioned non-traumatically overall by the arches 31 in the sinuses for radial and longitudinal positioning, while the lower support part 21 ensures longitudinal positioning on the ring, and the upper cylinder 11 prevents rocking. The result is that positioning on the ring is done without harm to the mitral valve.

Thus the invention makes it possible to make an artificial valve combining two parts, that is, a stent 1 and a valve 2, by sutures, staples, clips, or other means to form an overall structure that is very homogeneous and very strong, based on the interlacing of metallic and synthetic wires/threads.

In contrast to the artificial valves with a biological valve consisting of very fragile tissue, an artificial valve with a textile valve also makes it possible to steer clear of the problems of deterioration of the prosthetic material, problems essentially due to the prosthesis-metal interface, which can arise during the compression of the device before implantation.

Consequently, the invention favors the development of less onerous and less expensive surgical techniques—percutaneous implantation allowing for surgery that is easier for the patient.

Of course, the invention is not limited to the embodiment described and shown in the attached drawings. Modifications are possible, in particular from the standpoint of the composition of the various parts or by substitution of equivalent techniques, without leaving the scope of protection of the invention.

1.-25. (canceled)

26. Artificial valve characterized by the fact that it comprises: a stent or extensible armature (1) consisting of several parts, that is, an upper cylinder (11), a lower support part (21) in the form of a truncated cone whose maximum diameter is greater than that of the aortic annulus and decreases to the diameter of the stent or extensible armature (1) in the direction of the proximal end of the lower support part (21), which forms a partially spherical or toroidal surface, and three arches (31) placed at regular intervals in the lower part of the upper cylinder (11), which are connected to the upper cylinder (11), extend outward relative to the diameter of this latter, and deploy into the bulges formed by the sinuses, whereby the upper cylinder (11) is connected to the lower support part (21) by means of upright pieces (41); and a valve (2) consisting of a flexible membrane that is connected to the stent (1) by suturing, hooks, or clips.

27. Artificial valve according to claim 26, characterized by the fact that the arches (31) are attached to another part of the stent or extensible armature (1), that is, at the top or bottom of this latter, or else to the upright pieces (41).

28. Artificial valve according to claim 26, characterized by the fact that the upper cylinder (11) and the lower support part (21) in the form of a truncated cone are made by braiding and the arches (31) are also made by braiding and assembled by suturing to the upper cylinder (11), forming projections from this latter.

29. Artificial valve according to claim 26, characterized by the fact that the valve (2) is provided on the one hand with lips (2′) and on the other hand with a circular skirt (2″), whereby this circular skirt (2″) has the lips (2′) in its upper part and is folded like a conical cup (2′″), partly spherical or partly toroidal, along a fold line (2″″).

30. Artificial valve according to claim 26, characterized by the fact that the upright pieces (41) are attached at the top to the inside of the upper cylinder (11) of the stent (1).

31. Artificial valve according to claim 26, characterized by the fact that the upright pieces (41) are attached at the top to the outside of the upper cylinder (11).

32. Artificial valve according to claim 26, characterized by the fact that the upright pieces (41) are a solid part of the lower support part by having been braided, knitted, or machined into a single piece with this latter.

33. Artificial valve according to claim 26, characterized by the fact that there are six upright pieces (41), three of them ensuring that the upper cylinder (11) and the lower support part (21) are equidistant, and the other three ensuring that the valve (2) is attached to the lower support part (21).

34. Artificial valve according to claim 26, characterized by the fact that the upright pieces (41) are directly integrated at the bottom into the valve (2) during the production of this latter, in the form of metallic thread, thus creating a textile composite.

35. Artificial valve according to claim 26, characterized by the fact that the upper cylinder (11) and the lower support part (21) as well as the arches (31) of the stent (1) are made by interlacing metallic threads.

36. Artificial valve according to claim 35, characterized by the fact that the metallic threads are made of a material with shape memory.

37. Artificial valve according to claim 35, characterized by the fact that the metallic threads comprising the upper cylinder (11) and lower support part (21) as well as the arches (31) of the stent (1) are made of the same material or different materials.

38. Artificial valve according claim 26, characterized by the fact that upper cylinder (11) of the stent (1) is made of a pre-machined material with shape memory.
